(19) United States
(12) Patent Application Publication
(10) Pub. No.: US 2004/0193211 A1
(43) Pub. Date: Sep. 30, 2004

(54) Fingertip surgical instruments

(76) Inventors: James W. Voegele, Cincinnati, OH (US); Robert P. Gill, Mason, OH (US); Foster B. Stulen, Mason, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/777,324

(22) Filed: Feb. 12, 2004

Related U.S. Application Data
(60) Provisional application No. 60/447,446, filed on Feb. 14, 2003.

(51) Int. Cl. A61B 17/28

(52) U.S. Cl. 606/205

(57) ABSTRACT

Disclosed is a minimally invasive surgical instrument that may be used in hand-assisted laparoscopic surgeries. The device is multifunctional surgical instrument that may be mounted directly on a surgeon's fingertip and inserted through an incision to allow the surgeon to manipulate tissue during a surgical procedure.

### CROSS REFERENCE TO RELATED APPLICATIONS

The present application claims the benefit of U.S. Provisional patent application serial No. 60/447,446, filed on Feb. 14, 2003, the contents of which are hereby incorporated herein by reference.

The present application is also related to U.S. patent applications, attorney docket no. END-5015NP, Ser. No. [______] and END-5017NP, Ser. No. [______] filed concurrently herewith.

### FIELD OF THE INVENTION

The present invention relates in general to the performance of a variety of surgical steps or procedures during surgical operations and, more particularly, to methods and apparatus for utilizing fingertip surgical instruments as an integral part of such surgical procedures to expedite and facilitate the surgical procedure and to extend a surgeon's sense of “feel”.

### BACKGROUND OF THE INVENTION

Abdominal surgery typically involves an incision in the abdominal wall large enough to accommodate a surgeon's hands, multiple instruments, and illumination of the body cavity. While large incisions simplify access to the body cavity during a surgery, it also increases trauma, requires extended recovery time, and can result in unsightly scars. In response to these drawbacks, minimally invasive surgical methods have been developed.

In minimally invasive abdominal surgery, or laparoscopic surgery, several smaller incision are made into the abdominal wall. One of the openings is used to inflate the abdominal cavity with gas, which lifts the abdominal wall away from underlying organs and provides space to perform the desired surgery. This process is referred to as insufflation of the body cavity. Additional openings can be used to accommodate cannulas or trocars for illuminating and viewing the cavity, as well as instruments involved in actually performing the surgery, e.g., instruments to manipulate, cut, or resect organs and tissue.

While minimally invasive surgical methods overcome certain drawbacks of traditional open surgical methods, there are still various disadvantages. In particular, there is limited tactile feedback from the manipulated tissue to the surgeon hands. In non-endoscopic surgery, a surgeon can easily verify the identification of structures or vessels within a conventional open surgery incision. In particular the surgeon normally uses the sense of feel to verify the nature of visually identified operational fields. Further, in endoscopic surgery, tissue that is to be removed from the body cavity must be removed in pieces that are small enough to fit through one of the incisions.

Recently, new surgical methods have been developed that combine the advantages of the traditional and minimally invasive methods. It is sometimes referred to as hand assisted laparoscopic surgery (“HALS”). In these new methods, small incisions are still used to inflate, illuminate, and view the body cavity, but in addition, an intermediate incision is made into the abdominal wall to accommodate the surgeon's hand. The intermediate incision must be properly retracted to provide a suitable-sized opening, and the perimeter of the opening is typically protected with a surgical drape to prevent bacterial infection. A sealing mechanism is also required to prevent the loss of insufflation gases while the surgeon's hand is either inserted into or removed from the body cavity though the retracted incision.

While the hand provides a great deal of flexibility and retains the surgeon's sense of feel, fingers in themselves have limits as to their usefulness. Fingers lack the delicacy to pick up fine tissue. Fingers require making larger divisions when dissecting tissue. Fingers are subject to injury when holding tissue while energy modalities, such as ultrasound or RF, are used to treat the surgical site.

Traditional instruments intended for conventional surgery i.e. forceps and graspers are too large for the limited body cavity environment. Traditional instruments also present the problem of being brought into and out of the laparoscopic site causing time-delaying deflation and re-insufflations of the body cavity. Laparoscopic equivalent instruments are delivered through a body wall port and have limited access to tissue.

U.S. Pat. Nos. 5,42,227; 6,149,642; 6,149,642; 5,925,064 disclose various aspects of laparoscopic surgery and fingertip devices for surgeon use.

With the advance represented by HALS procedures there is a need for improved fingertip surgical instrumentation that can take advantage of the increased freedom created by having a hand inside the body cavity. The present invention overcomes the disadvantages of the prior art and provides the surgeon with a cost effective, yet efficiently flexible surgical instrument.

### BRIEF SUMMARY OF THE INVENTION

This need is met by the methods and apparatus of the present invention wherein an a surgical device defined by attachment to a surgeon's hand such that it is used to operate within an operational field.

### BRIEF DESCRIPTION OF THE FIGURES

These and other features, aspects, and advantages of the invention will become more readily apparent with reference to the following detailed description of a presently preferred, but nonetheless illustrative, embodiment when read in conjunction with the accompanying drawings. The drawings referred to herein will be understood as not being drawn to scale, except if specifically noted, the emphasis instead being placed upon illustrating the principles of the invention. In the accompanying drawings:

FIG. 1a is a cut-away perspective view of an exemplary use of the present invention;

FIG. 1b is a cut-away view of one embodiment of the invention attached to a surgeon's finger;

FIG. 2 is a perspective of one embodiment of the invention attached to a surgeon's fingertip;

FIGS. 3a is a perspective view of one embodiment of the invention having a scissors working element and a pushbutton actuation mechanism;

FIG. 3b is a cut-away elevation view of the pushbutton actuation mechanism of FIG. 3a;

FIG. 3c is a perspective view of a one-finger operation scissors working element;

FIG. 3d is a perspective view of a two-finger operation scissors working element;

FIGS. 4a-b are perspective views of alternate embodiments of the invention having a tissue grasper working element;

FIG. 5 is a perspective view of an alternate embodiment of the invention having a clip applier working element;

FIGS. 6a-c are a perspective views of alternate embodiments of the invention RF-energized working element;

FIGS. 7a-f are perspective views of an alternate embodiment of the invention having a monopolar working element that are interchangeable;

FIG. 8 is a perspective view of an alternate embodiment of the invention having a tissue grasper working element and a thumb-actuated closure mechanism;

FIG. 9 is a perspective view of an alternate embodiment of the invention having a suction/irrigation working element;

FIG. 10a is an elevation view of an alternate embodiment of the invention having a tissue grasper working element and a spring-biased moveable jaw;

FIG. 10b is a cut-away elevation view of the embodiment of the invention shown in FIG. 10a;

FIG. 11 is a cut-away elevation view of an alternate embodiment of the invention having a needle holder working element;

FIGS. 12a-d are alternate views of an alternate embodiment of the invention having a right angle dissector working element;

FIG. 13a-c are alternate views of an alternate embodiment of the invention having a scissors working element;

FIG. 14a is a cut-away perspective view of an exemplary use of the present invention having a ultrasonic working element;

FIG. 14b-c are views of a representative transducer assembly for use in the embodiment of FIG. 14a; and

FIG. 14d is a perspective view of a exemplary transducer and blade assembly for use in the embodiment of FIG. 14a.

### DETAILED DESCRIPTION OF THE INVENTION

Before explaining the present invention in detail, it should be noted that the invention is not limited in its application or use to the details of construction and arrangement of parts illustrated in the accompanying drawings and description.

The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, expressions of embodiments, examples, methods, etc. can be combined with any one or more of the other following-described embodiments, expressions of embodiments, examples, methods, etc.

While the methods and apparatus of the present invention are generally applicable to the performance of these surgical procedures during any operation, they are particularly applicable to their performance during HALS procedures and, accordingly, will be described herein with reference to this invention.

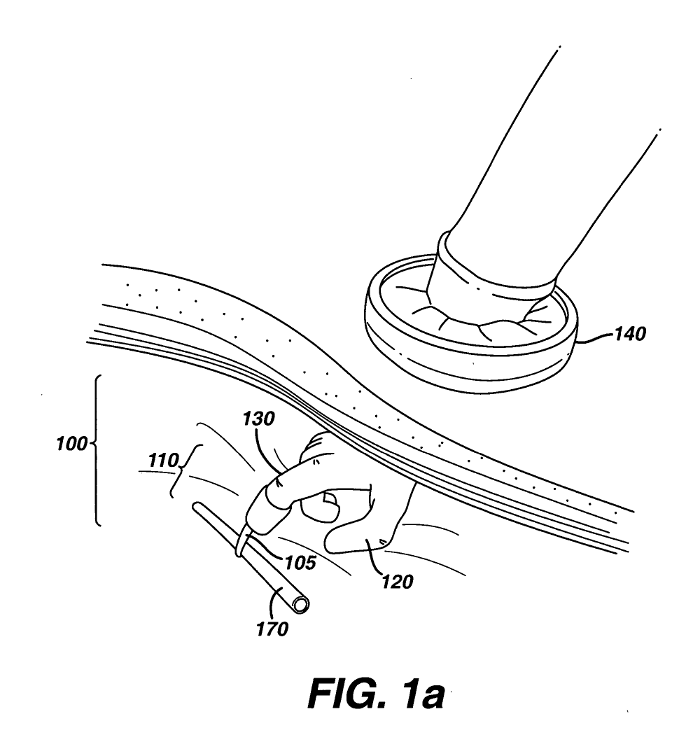

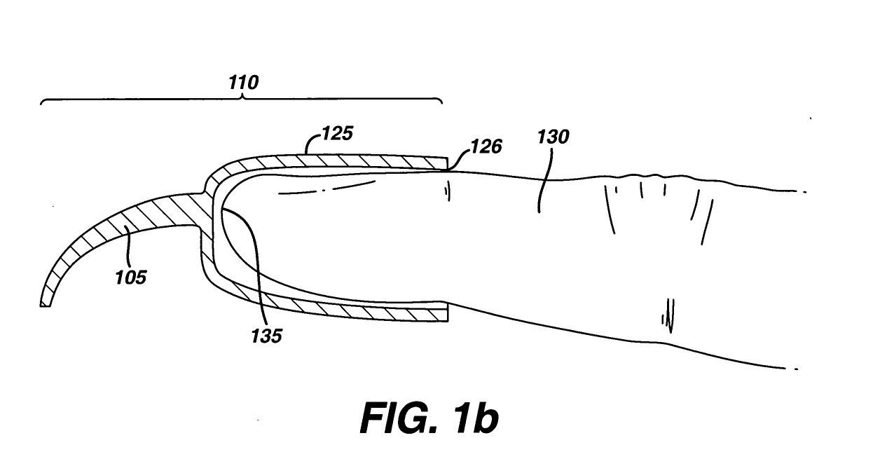

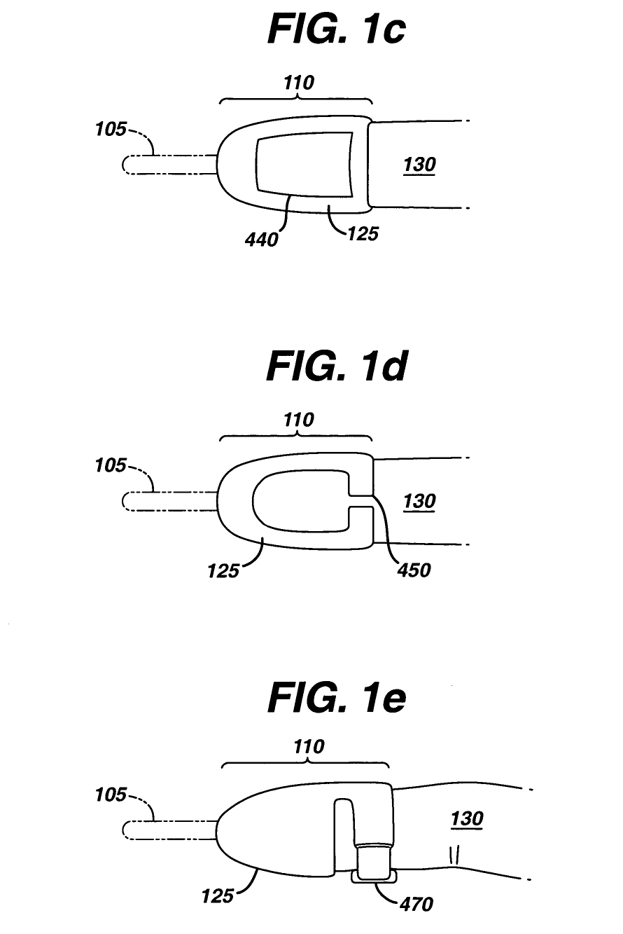

Referring now to FIG. 1a, the environment for performing an endoscopic surgical procedure within an abdomen 100 is illustrated. A means for providing hand access, such as a lap disc 110, for example, model LD111 available from Ethicon Endo-Surgery, Cincinnati, Ohio, is placed into the abdominal wall. A surgeon places his arm and gloved hand 120 through the lap disc and into the abdomen cavity 100. The index finger 130 (although any finger can be used) is capped with a finger device with a surgical instrument 110 having (in a generic sense) a working element 105. The working element 105 can be used to manipulate tissue, such as for example, a blood vessel 170 during a laparoscopic procedure.

FIG. 1b is a cut-away view of a fingertip instrument 110 having a finger-insert member or shell 125 defining a cavity 126 for releasably receiving a finger 130 fully inserted into the shell 125 with fingertip 135 resting at the distal end of cavity 126. Preferably, shell 125 and cavity 126 are constructed to compressively engage the surgeon's fingertip 135. Cavity 126 may also have a friction material on its internal surface to provide further gripping capabilities to secure the surgeon's fingertip 135. Shell 125 may also comprise a mounting means (not shown), such as a strap, to securely fasten the shell 125 to the surgeon's finger 130. Fingertip instrument 110 may be reusable or disposable and made from a biocompatible material such as plastic or stainless steel. Working element 105 may be constructed from a plastic or stainless steel depending upon its particular function as is described in more detail below.

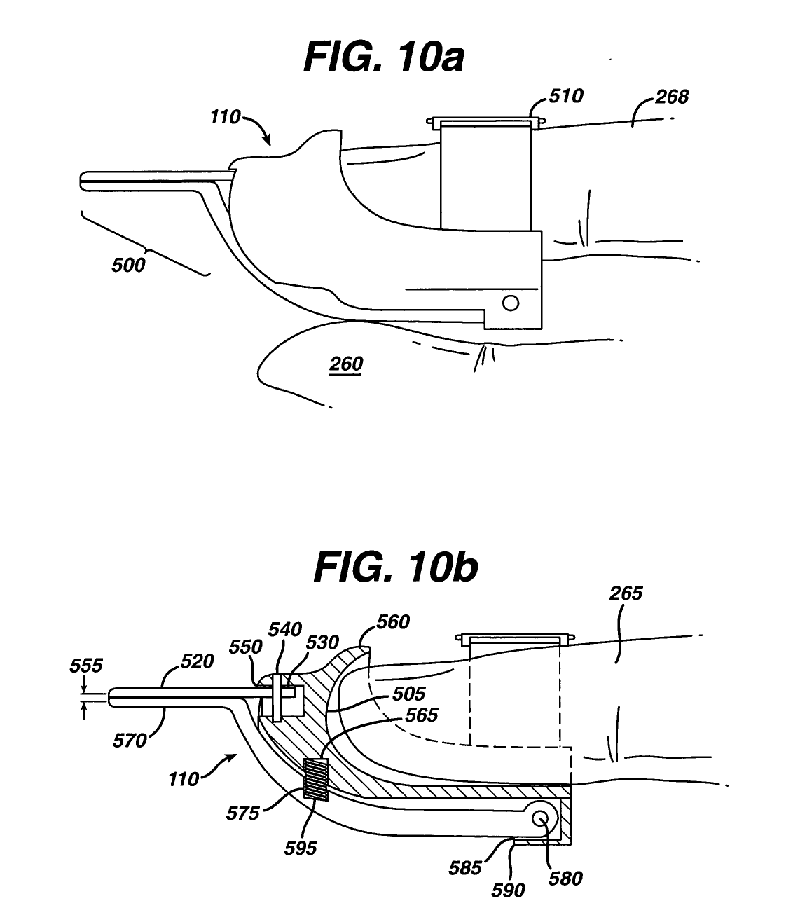

FIGS. 1c-d illustrate alternate configurations of shell 125 to meet varying surgeon requirements and sizes of fingers. FIG. 1c is a side view of shell 125 illustrating an opening 440 to enable the surgeon to feel tissue while fingertip instrument 110 is attached. FIG. 10d is useful to accommodate varying finger sizes by providing a rim break 450 to allow shell 125 to deflect thereby fitting a greater range of finger sizes. FIG. 1e illustrates a two-piece snap band 470 that overlaps and snaps in place to accommodate finger size variations. Other configurations of shell 125 embodies side walls of a flexible nature i.e. elastomer or weave pattern that allow the Instrument 110 to be folded to enable its delivery into the body cavity through other devices, such as a trocar.

Alternate embodiments of the fingertip devices incorporate an adjustable strap to accommodate a greater finger size range. The profiles have also been adapted to enable alternate actuation means.

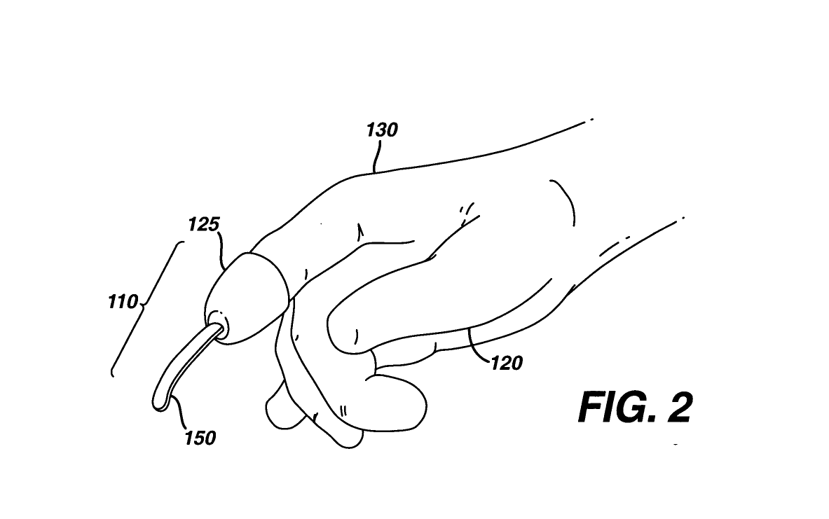

FIG. 2 is a perspective of instrument 110 having a blunted working element or extension tip 150 protruding from the distal end of finger insert member 125.

Extension tip 150 can be conveniently used for non-sharp piercing, elevating or dividing tissue.

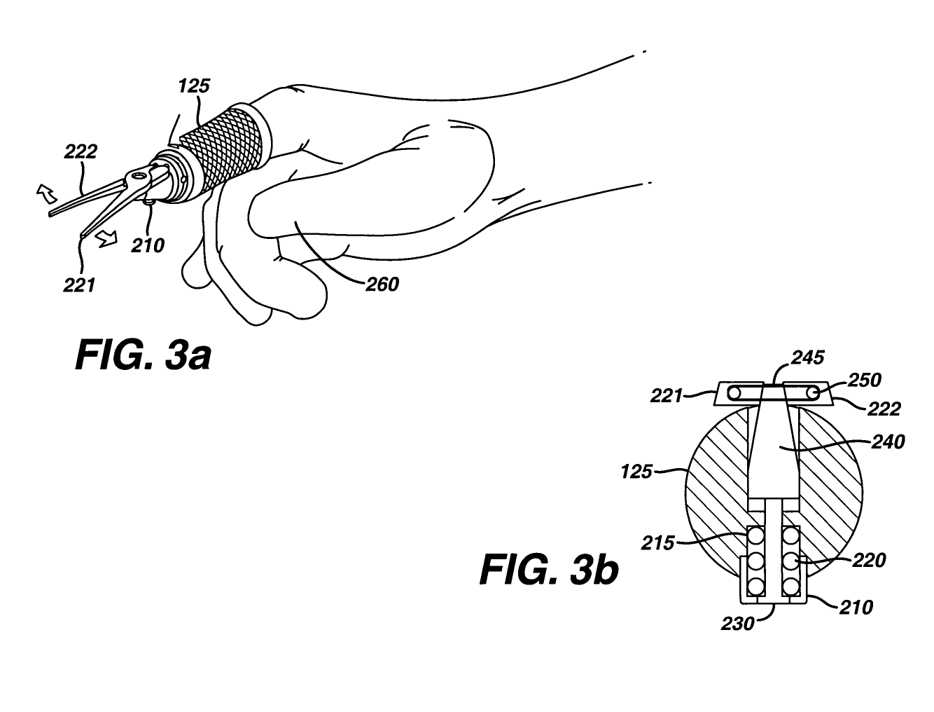

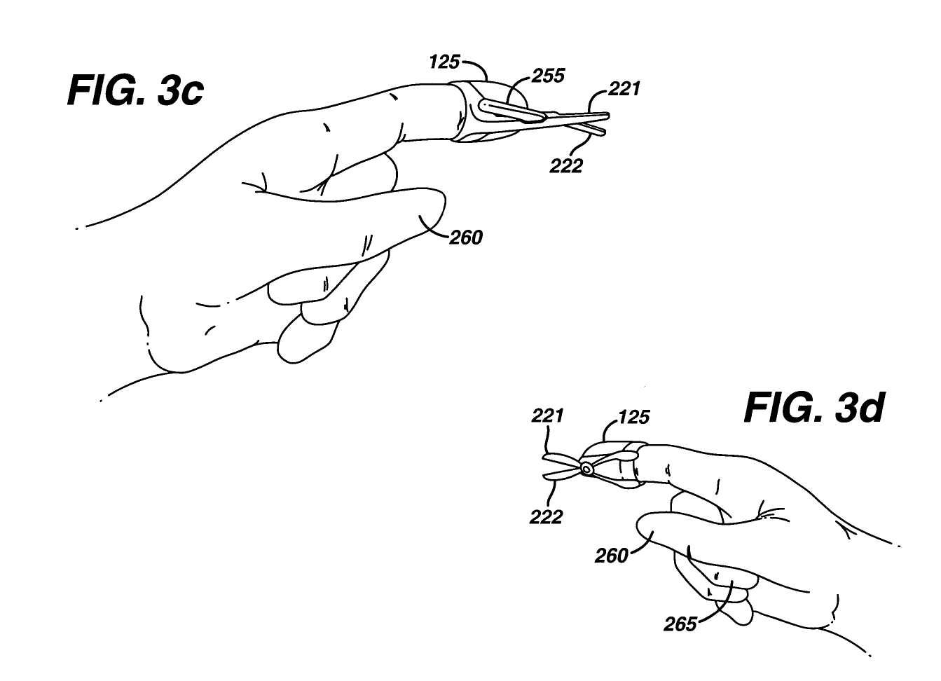

FIG. 3a-d illustrate a third embodiment of a fingertip surgical instrument 125 having a working element defining a scissors element. FIG. 3a illustrates a single finger operated scissors having a spring loaded push button 210 driving scissor halves 222 and 221 apart from each other. FIG. 3b shows a cross section of button 210 mechanism consisting of wedge Shaft 240 that connects to the button 210 at joint 230. Wedge shaft 240 is captured within the pocket 215 cut into shell 125. By pressing button 210, spring 220 compresses driving the wedge 240 between scissor halves 221, 222 that have an elastic band 245 stretched between posts 250 to apply a return force. FIG. 3c illustrates a one-finger operation fingertip instrument having a scissors working element. A scissor half 221 is fixed to the shell 125 and the other scissor half 222 is operable by moving thumb lever 255. FIG. 3d illustrates a two-finger operated working element where the thumb 260 and other finger 265 operate lever arms associated with scissor halves 221, 222.

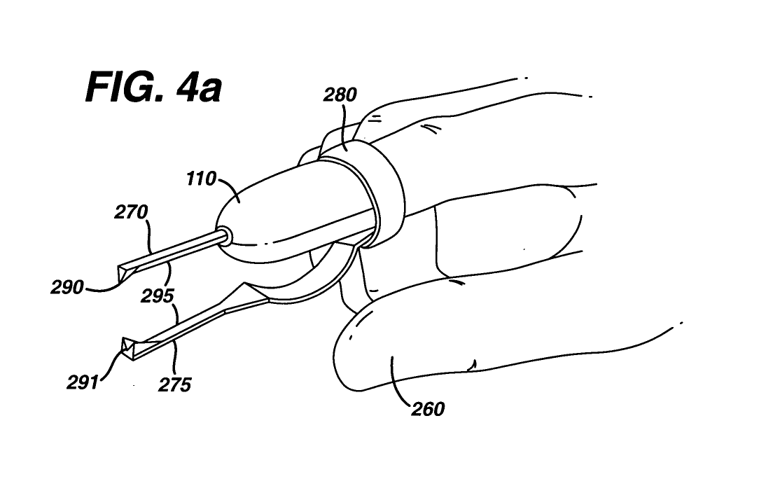

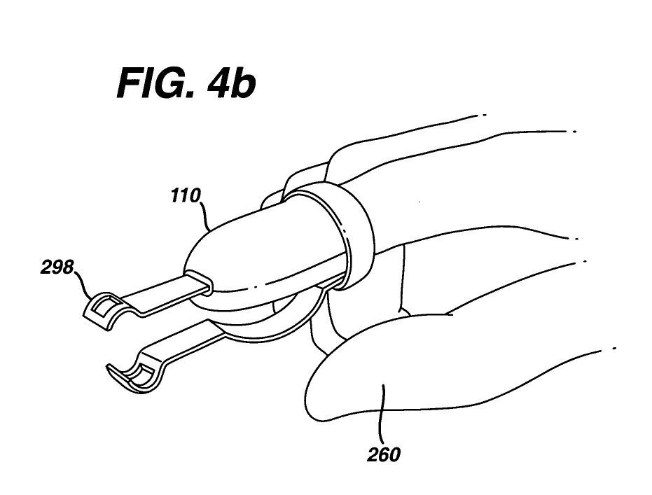

FIGS. 4a-b illustrate a fourth embodiment of fingertip instrument 110 having a tissue pick-up working element. In FIG. 4a, a stationary arm 270 opposes a flexible arm 275 attached to shell 110 by a rigid band 280. Thumb 260 actuates the flexible arm 275 to engage tissue between teeth 290 and 291. Teeth 290 and 291 may have any variety of tissue grasping configurations, such as interlocking or serrated. FIG. 4b illustrates a Babcock shape 298 as an example of the many other applicable well known forms.

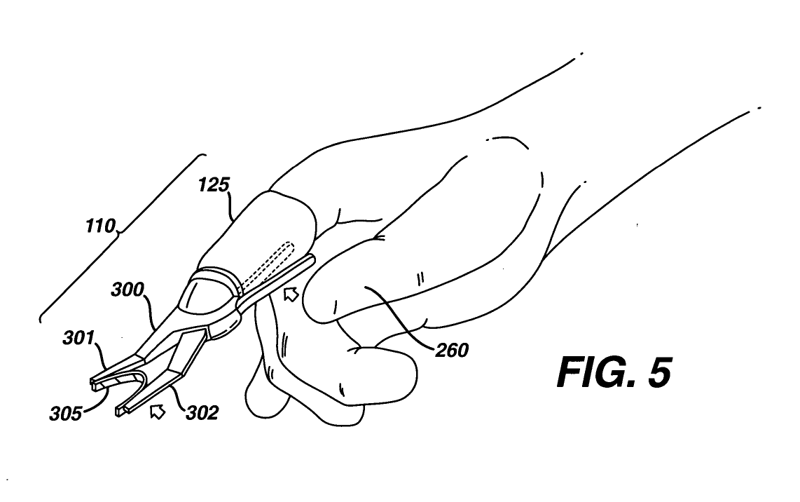

FIG. 5 illustrates a fifth embodiment of fingertip instrument 110 having a clip applier working element. Frame 300 consists of a stationary jaw 301 and a moveable jaw 302, which is actuated by lever 260. Jaws 301 and 302 are configured to hold a clip 305. The surgeon may navigate clip 305 around tissue or a blood vessel and actuate lever 260 to deform clip 305 around the tissue.

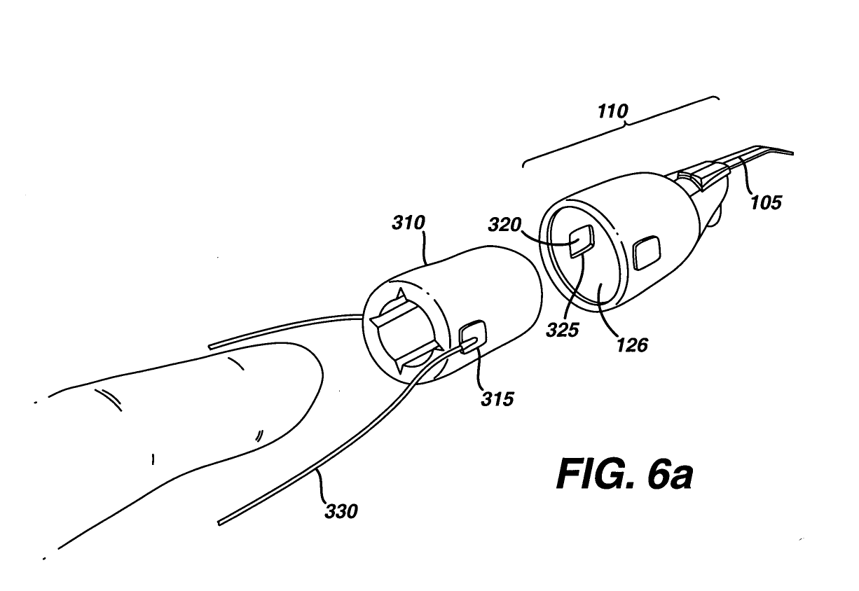

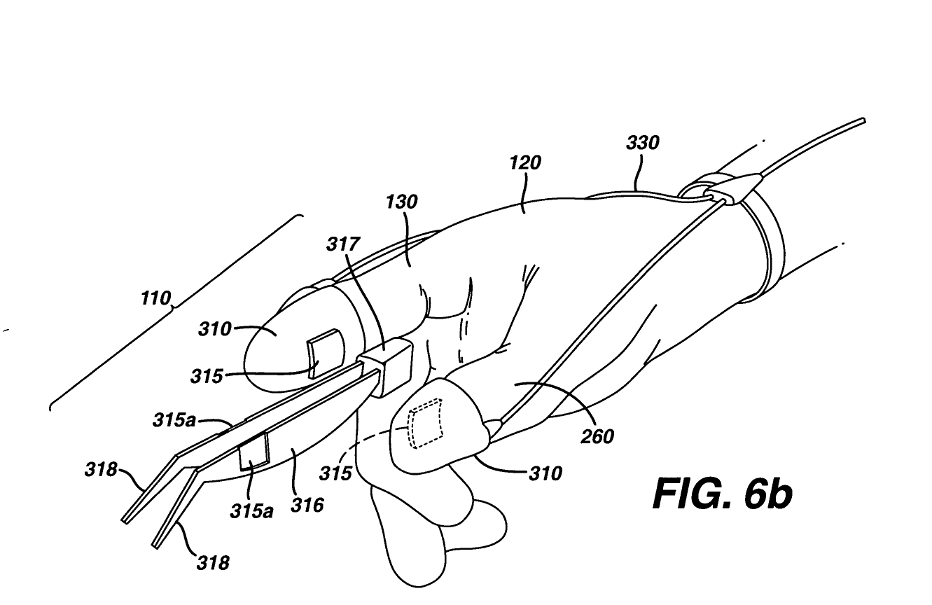

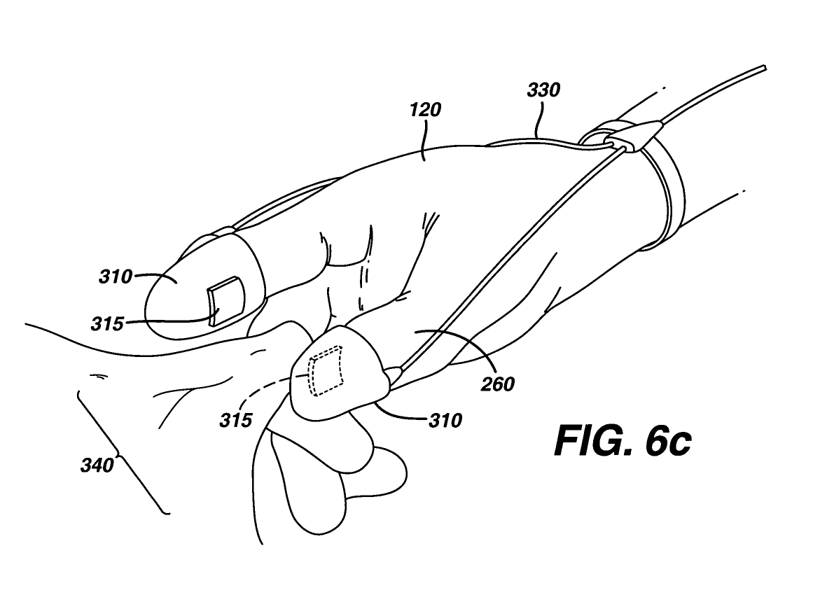

FIGS. 6a-c illustrate a sixth embodiment of fingertip instrument 110 having an RF working element. FIG. 6a illustrates an electrical insulating conformable RF finger cuff 310 containing electrodes 315. Fingertip instrument 110 with working element 105 slips over finger cuff 310 and electrodes 315 mate with contacts 320 contained within the cavity 126 of instrument 110. FIG. 6b illustrates two electrodes 315 contained on the thumb and index finger, for example, that interface with an RF pick-up or bipolar forceps 316 via contacts 315a and applying an insulator 317 between the two tissue contacting elements 318. FIG. 6c discloses a bipolar application using two RF finger cuffs 310, one electrode 315 on index finger 130 and one electrode 315 on thumb 260. In this manner, RF energy would be directly applied to tissue 340. In each of the described embodiments, RF energy is provided to the finger cuffs via wires, that may be, for example, attached to the surgeon's arm and connected to a standard RF generator. The delivery of RF energy to the finger cuffs would be controlled by an external means such as a foot pedal (not shown). In all cases, the RF applications may be monopolar with one electrode and a grounding pad (not shown) or bipolar.

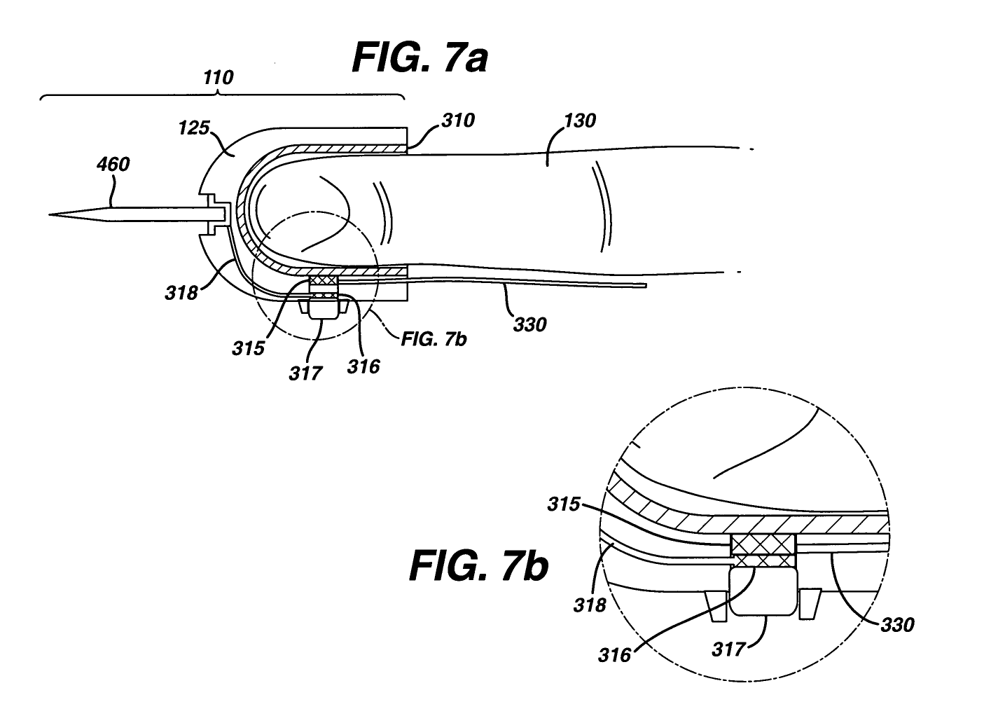

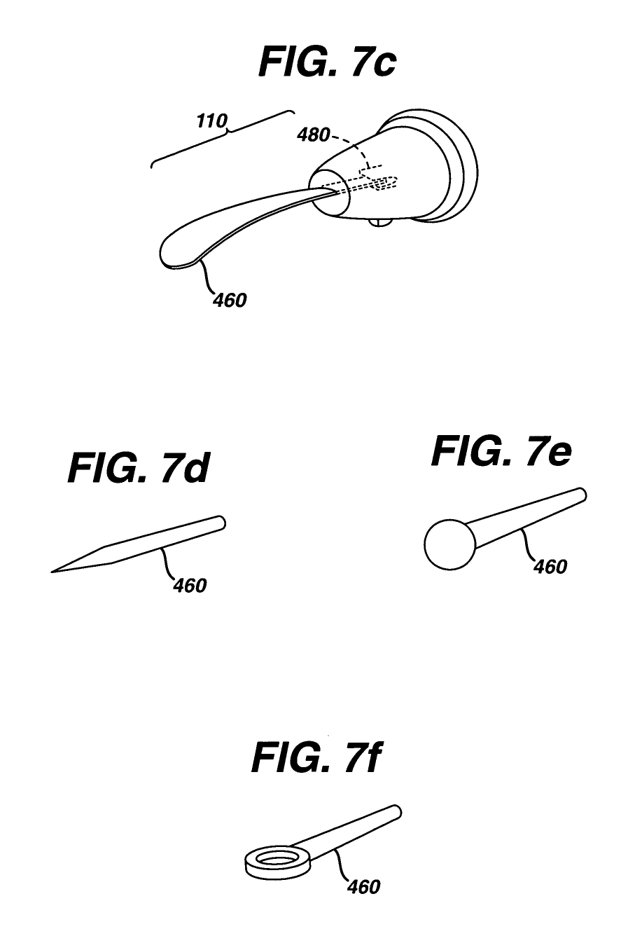

FIGS. 7a-f illustrate a seventh embodiment of the fingertip instrument 110 having a monopolar working element 460. In this embodiment, an insulated finger cuff 310 comprises an electrode 315 connected to an RF generator via conductor 330. Finger cuff 310 inserts within shell 125 and electrode 315 interfaces with contact 316 that is mechanically connected to button 317. Contact 316 electrically connects with monopoloar working element 460 via conductor 318 molded within shell 125. Button 317 may be any number of conventional mechanical devices for causing contact 316 to make electrical contact with electrode 315 (FIG. 7b). Button 317 enables the surgeon to activate working element 460 via thumb. Thumb 160 (not shown) to activate the Tip Electrode 460 if a hand switch is desired. As would be apparent to those skilled in the art monopolar working element 460 may also be configured for bipolar operation including cut and coagulation operation. In another instance, working element 460 may be removably attached to shell 125 to allow for multiple working elements to be used without having to change finger tip instrument 110. Working element 460 may interface with conductor 318 via a contact terminal 480 positioned within shell 125. Other possible working elements 460 are illustrated in FIGS. 7d-f.

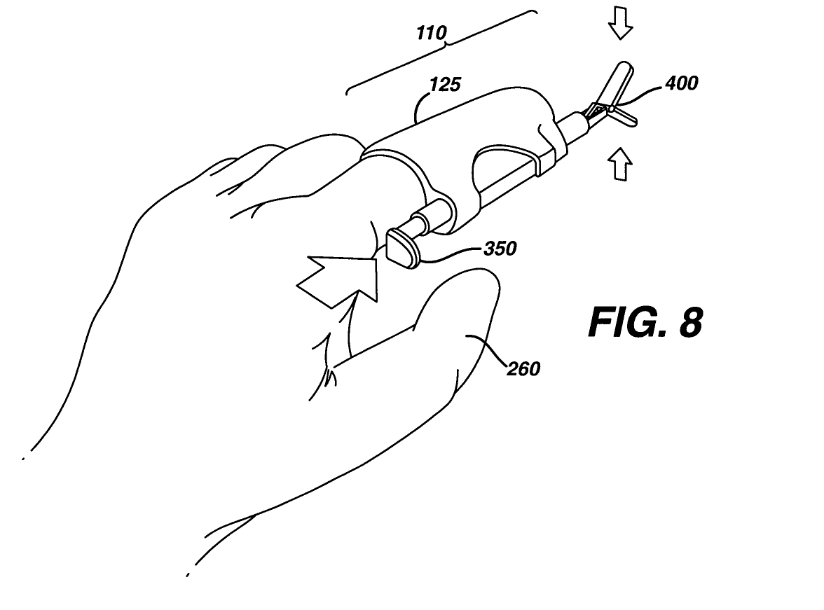

FIG. 8 illustrates an eighth embodiment of the fingertip instrument 110 having a grasper working element 400. Grasper 400 has two moveable jaws that are controlled via a thumb-actuated push button 350 for activating grasper 400. In one instance push button 350 may activate an actuation tube as part of tube-in-a-tube construction, well known to those skilled in the art, to cause the jaws of grasper 400 to grab and release tissue.

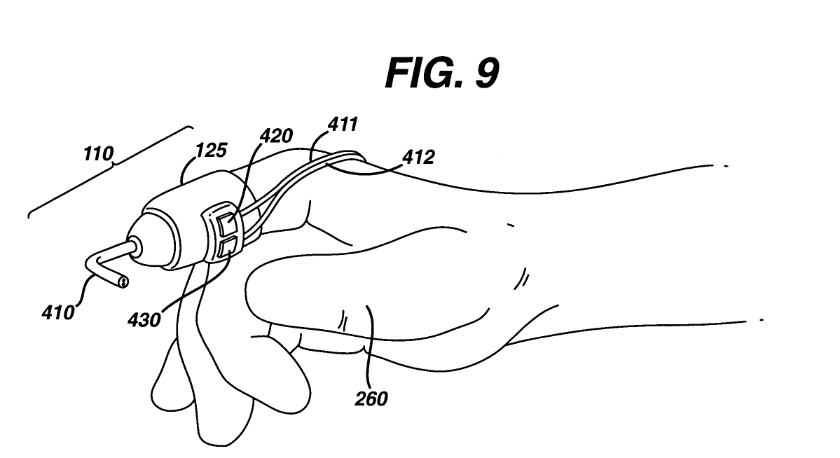

FIG. 9 illustrates a ninth embodiment of the fingertip instrument 110 having a suction/irrigation working element 410. Suction and irrigation lines 411 and 412 travel from a standard suction/irrigation supply via the surgeon's arm and terminate at corresponding actuation buttons 420 and 430. The surgeon may selectively manipulate working element 410 within the operation site and cause fluid suction or irrigation via thumb 260 actuation as required during the medical procedure.

FIG. 10 illustrates a tenth embodiment of the fingertip instrument 110 having a tissue forceps 500 as a working element. As shown in FIGS. 10a-b, tissue forcep 500 comprises a stationary jaw 520 and a moveable jaw 570 that is acutated by a thumb 260. FIG. 10 also illustrates an alternate configuration of shell 560. In this instance shell 560 is open in design and a mechanical fastener, such as a strap 510, securely fastens shell 560 to finger 265.

Referring to FIG. 10b, stationary jaw 520 has block end 530 that is secured by a stationary jaw pin 540 or equivalent cross member into a body recess 550 of the shell 560. The movable jaw 570 rotates about a pivot pin 580 at the proximal end of the jaw 570. Jaw 570 is spring biased away from shell 560 by means of spring 575 positioned within recess 565. Ledge 590 acts as a stop for jaw 570 and clearance 585 determines the maximum jaw opening 555 when jaw 570 is fully retracted.

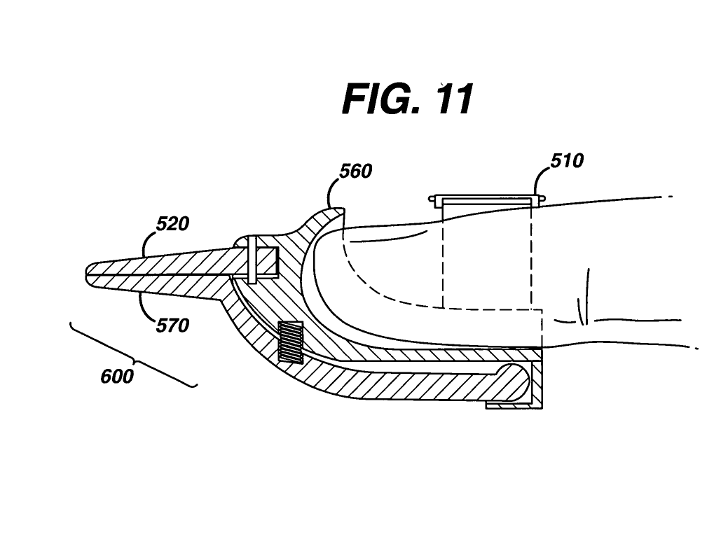

FIG. 11 is an alternate working element in the form of a needle holder 600 in conjunction with the embodiment of FIG. 10. Needle holders 600 may also include a ratcheting mechanism well known to the instrument making art to accommodate varying needle sizes and/or clamping pressures (not shown).

Generally, the working element may take any number of configurations that are readily observable in surgical catalogs, for example, the Codman Surgical Product Catalog, Division of Johnson and Johnson, New Brunswick, N.J.

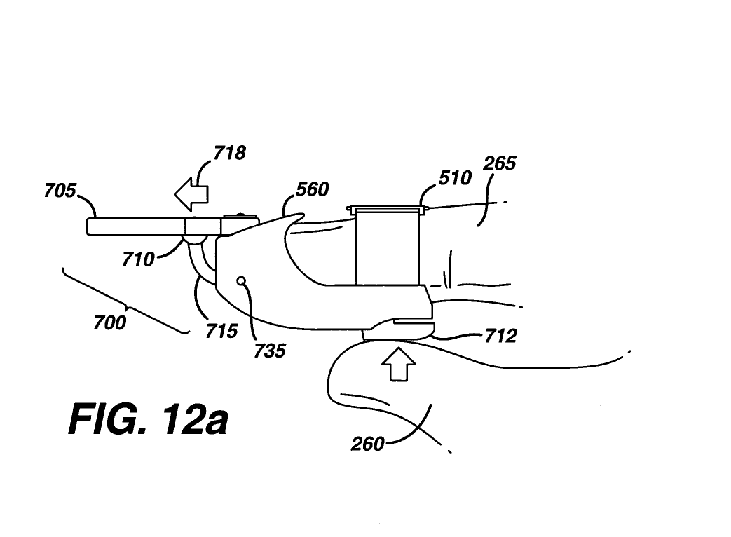

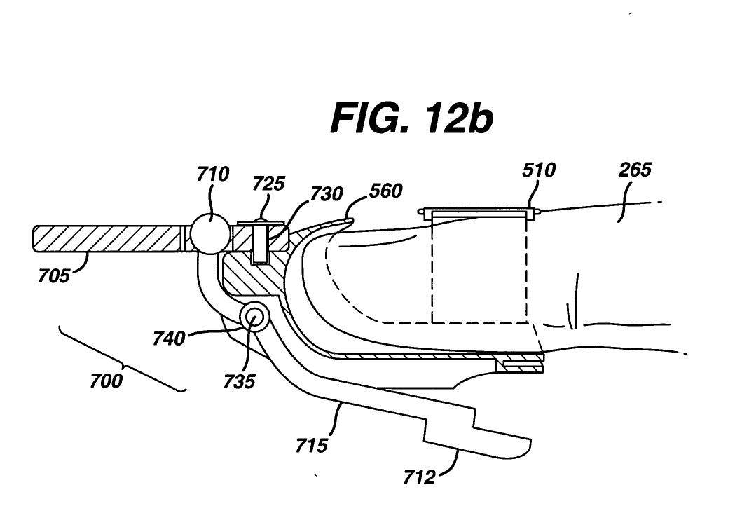

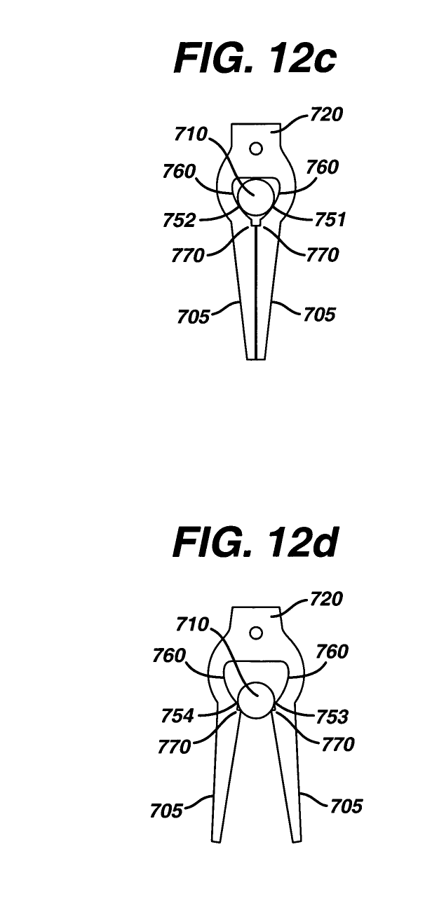

Referring to FIGS. 12a-d a right angle dissector 700 is shown. Jaws 705 are caused to spread when the actuator ball 710 is moved from a first position (FIG. 12c) distally to a second position (FIG. 12d). Jaws 705 emanate from a common end 720 that is secured to the shell 560 by a pin 725 that is anchored into a mating pin recess 730 of shell 560. An actuation arm 715 is connected to shell 560 via pivot pin and concentric pivot hole 740. Surgeon thumb 260 actuates pivot arm 715 via thumb pad 712. When pivot arm 715 is actuated, ball 710 is forced distally and spreads jaws 705 and initial ball contact points 751, 752 move to diametric tangential positions 753, 754 as ball 710 slides along the surface faces 760 to achieve the maximum jaw spread 765. The jaws 705 may have a surface break 770 that enables ball 710 to stay in its most distal position without having the surgeon maintain constant pressure on the thumb pad 712.

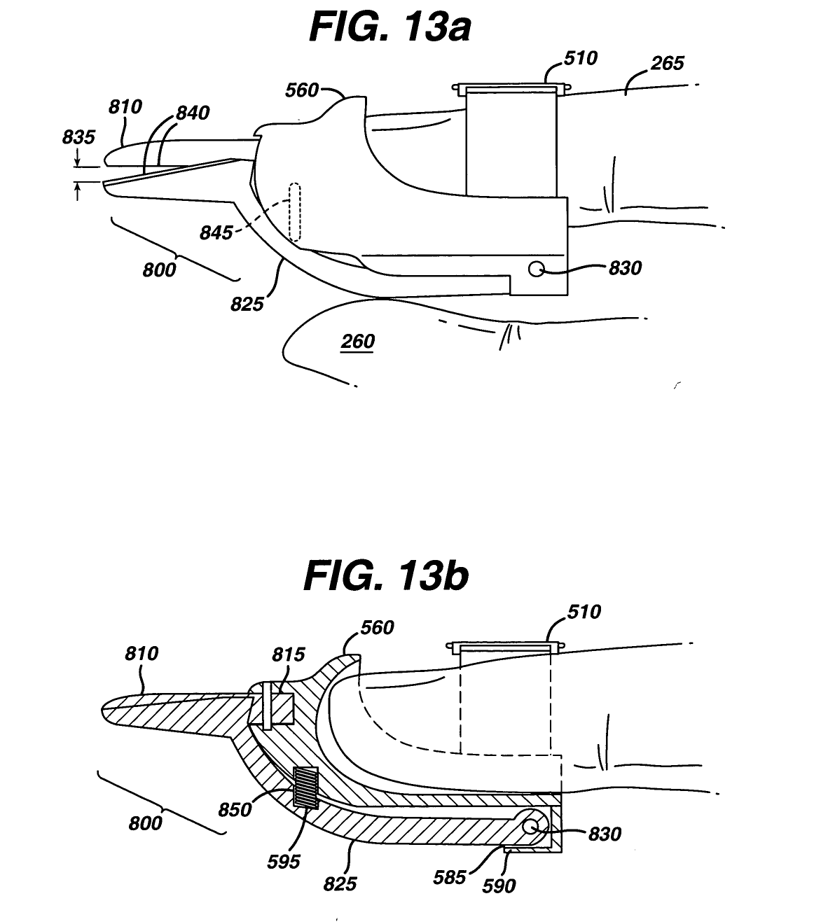

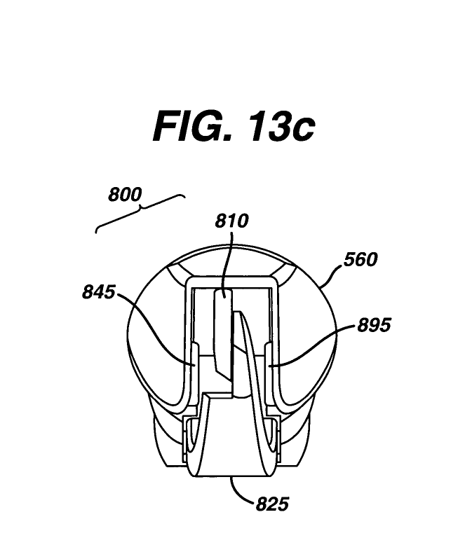

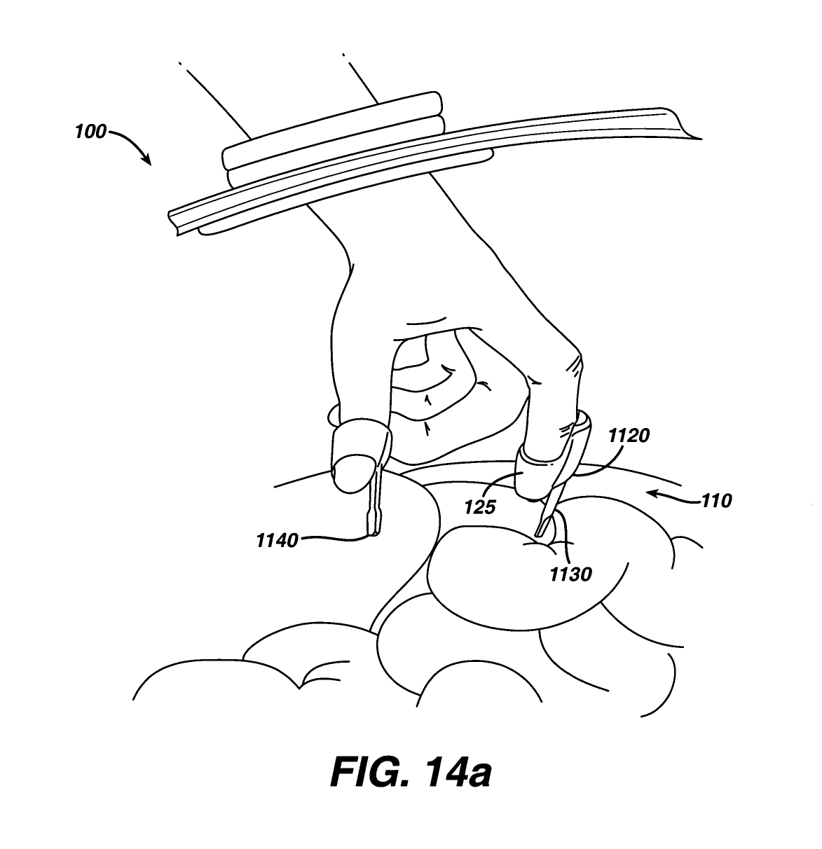

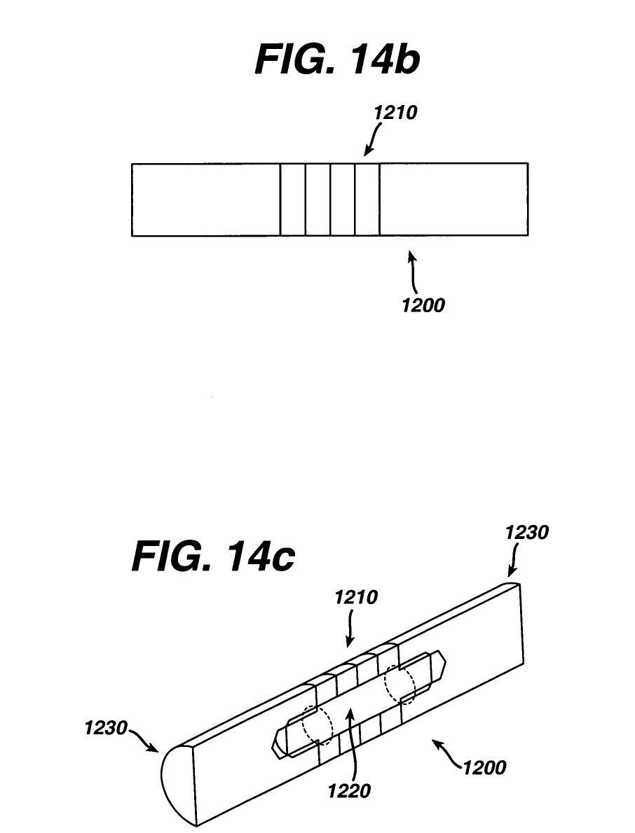

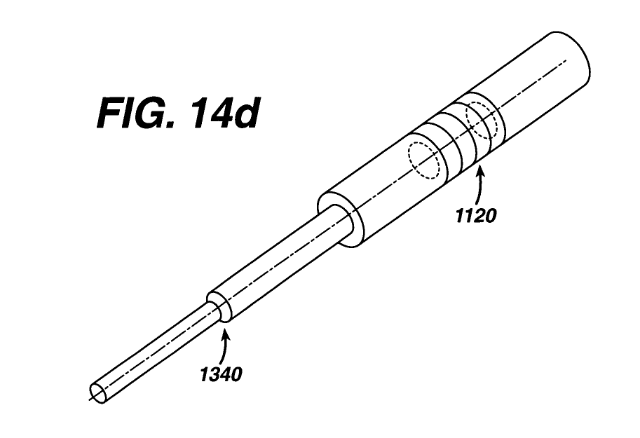

FIGS. 13a-c represent still an alternate embodiment of a working element in the form of a scissors in conjunction with the embodiment of FIG. 10 with like reference numerals having the same function. Scissor working element 800 includes a stationary jaw 810 and a moveable jaw 825. The cutting faces 840 (FIG. 14a) are contoured to established industry standards for tissue cutting performance. To prevent the cutting faces 840 to separate and leaving gaps in the resulting tissue cut, a raised rib 845 assist the intended alignment of the moveable scissor jaw 825 with respect to stationary jaw 810.

FIGS. 14a-d illustrates an alternate embodiment of the fingertip instrument 110 having an ultrasonic scalpel or blade 1130 as a working element. The ultrasonic instrument includes a transducer section 1120 that is molded or otherwise housed into the finger shell 125 and a blade 1130 that attaches to the transducer 1120 and extends distally to contact and manipulate tissue. A cable, not shown, extends from the instrument back along the hand and arm through the hand port 100 to an ultrasonic generator.

The ultrasonic blade 1130 is envisioned as a spatula or spoon-like device as depicted in FIG. 14a. The instrument can be used without ultrasonic energy for fine dissection and creating planes. With ultrasound energy applied, the blade can be used to cut and close small bleeders by pressing against them.

A second instrument 1140 has a passive tine that would be mounted with another finger shell or ring to the thumb as depicted in FIG. 14a. Together the thumb and index finger instruments can be used as a pair of tissue pickups. In this configuration, they are a natural extension to pick up items/tissue between the index finger and the thumb. With the ultrasonic energy activated the two instruments would act like a pair of RF-bipolar forceps. However, the ultrasonic fingertip forceps provide the benefits of ultrasound: minimal lateral thermal damage, less stick and char, no stray electrical currents, coagulation and transection in one application, and multi-functionality.

Another embodiment not shown incorporates the passive tine and ultrasonic active tine into one finger shell instrument similar to the embodiments shown in FIGS. 10-12. The instrument would likely be placed on the index finger. The thumb would be used to press the passive tine onto the active tine. Again with out ultrasound, the forceps would act as a simple tissue pick-up to aid in dissection. With the ultrasound applied, the forceps would be used to coagulate and transect small vessels.

The ultrasonic transducer in 1120 is designed as a conventional Langevin bolted transducer well known by those practicing in the art. The actual ultrasonic transducer 1200 shown in FIG. 14b consists of a stack of piezoelectric disks 1210 connected to metallic ends 1230, referred to as end masses. The piezoelectric elements are driven by a generator that tracks the desired resonant frequency as it changes with temperature and load and also supplies electrical power at the resonant frequency. The electrical energy is transformed into ultrasonic energy by the piezoelectric elements.

The piezoelectric elements contract and expand creating alternating periods of compression and tension. Because common piezoelectric materials are ceramics, they are weak in tension. Therefore the piezoelectric elements are pre-compressed by a bolt that is generally tightened between the two metallic end mass. The center bolt 1220 is shown in FIG. 14c as engaging threads in both end masses 1230. Often the center bolt passes through one end mass and through the center of the piezoelectric elements that are typically ring-shaped.

The shank of the bolt engages threads in the opposite end mass and tightened to apply the pre-compression.

The transducer 1120 is sized to be on the order of the distal and middle phalanges of the index finger. The length is on the order of two inches or less and the diameter should be nominally ½ inch or less. The actual length and diameter depend on the selected frequency of operation, number of piezoelectric elements, metals used in the end masses, size of compression bolt, and other design specifics.

The transducer could be designed as either a ¼ wavelength or a ½ wavelength. The transducer could be designed with more ¼ wavelengths, but a goal in this application it to keep the transducer small and non-intrusive. The ¼ wavelength design has all of the piezoelectric elements to one side of a vibration node. The end mass near the node is relatively short in length. It is still necessary to accept the pre-compression bolt and to mount the blade possibly with the threads of the bolt extending through the thin end mass. A ½ wavelength transducer would have nominally equal end masses. The piezoelectric elements would be centrally located with an equal number on either side of the displacement node.

For example, a symmetric ½ wavelength transducer design 1200 is shown in FIG. 14b-d. Four piezoelectric elements 1210 are centered along the transducer. The piezoelectric material used in this design is PZT-8 available from several piezoelectric suppliers. The center bolt 1220 extends through the piezoelectric elements and is attached to the two end masses 1230. The end masses 1230 are made from a titanium alloy (Ti6AI4V). The overall length is 1.58 inches, and the diameter is 0.3 inches. The maximum power is estimated to be on the order of about 25 watts.

In order to achieve higher displacements, ½ wave resonator sections are typically attached to a transducer. These resonators can be designed to supply displacement gain. Therefore, the blade portion is designed as a half wave resonator. Gain is supplied when the diameter of the proximal ¼ wavelength is greater than the distal ¼ wavelength. When the proximal and distal ¼ wavelengths have uniform cross-sections (not necessarily the same cross sections) and the change in the area occurs in the center, then the gain is determined by the ratio of the areas. So for example, if the distal section has half the area of the proximal section then the gain is 2.0. The displacement node is also at the step change. Different features, such as a spatula like end will change the gain and nodal location. But determination of the gain and nodal location for a particular design in the art is well known by those practiced in the art.

A simple blade 1340 with out a spatula end is shown attached to transducer 1200 in FIG. 14d. The blade is composed of two cylindrical ¼ wavelength sections. The ratio of the proximal area to the distal area is 2.5, so that the gain is nominally 2.5. Greater gains can be achieved by increasing the area ratio, adding some gain in the transducer section, or with the addition of ½ wavelength to the blade with gain.

While preferred embodiments of the present invention have been shown and described herein, it will be obvious to those skilled in the art that such embodiments are provided by way of example only. In addition, it should be understood that every structure described above has a function and such structure can be referred to as a means for performing that function. Numerous variations, changes, and substitutions will now occur to those skilled in the art without departing from the invention. Accordingly, it is intended that the invention be limited only by the spirit and scope of the appended claims.

What is claimed is:

1. A fingertip-mounted minimally invasive surgical instrument comprising:
a) a finger mount, having a proximal and distal end, and a cavity for releasably receiving a fingertip; and
b) a working element extending from the distal end of the finger mount.

2. The fingertip-mounted minimally invasive surgical instrument of claim 1, wherein the working element is a scissors element having a stationary jaw and a moveable jaw.

3. The fingertip-mounted minimally invasive surgical instrument of claim 1, wherein the working element is a tissue grasper.

4. The fingertip-mounted minimally invasive surgical instrument of claim 1, wherein the working element is a clip applier.

5. The fingertip-mounted minimally invasive surgical instrument of claim 1, wherein the working element is connected to an RF energy source.

6. The fingertip-mounted minimally invasive surgical instrument of claim 1, wherein the working element is a blade connected to an ultrasonic transducer.

7. The fingertip-mounted minimally invasive surgical instrument of claim 1, wherein the working element is an aspirator and suction element.

8. A method of performing a minimally invasive surgical procedure in a patient comprising:
a) creating an incision to permit hand access within the patient;
b) introducing a hand instrument comprising:
i) a finger mount, having a proximal and distal end, and a cavity for releasably receiving a fingertip; and
ii) an ultrasonic transducer positioned on the finger mount and a blade extending distally from the transducer; and
c) actuating the transducer to deliver ultrasonic energy to the blade.

9. The method of claim 8 further comprising the step of releasably engaging a finger with the hand instrument.

10. The method of claim 8 further comprising the step of actuating the transducer to provide therapeutic effects to the surgical site.
